(19) United States
(12) Patent Application Publication
(10) Pub. No.: US 2026/0219094 A1
(43) Pub. Date: Jul. 30, 2026

(54) URINATION AMOUNT MEASURING APPARATUS

(71) Applicant: SHIMADZU CORPORATION, Kyoto-shi, Kyoto (JP)

(72) Inventors: Katsumi SEKIKAWA, Kyoto-shi, Kyoto (JP); Kazunori SHIMIZU, Kyoto-shi, Kyoto (JP); Yuri ITO, Kyoto-shi, Kyoto (JP)

(73) Assignee: SHIMADZU CORPORATION, Kyoto-shi, Kyoto (JP)

(21) Appl. No.: 19/148,806

(22) PCT Filed: Nov. 28, 2023
(86) PCT No.: PCT/JP2023/042526

§ 371 Date: Jul. 17, 2025

(30) Foreign Application Priority Data
Jan. 30, 2023 (JP) 2023-012196

(51) Int. Cl. G01G 17/04; A61B 5/20; G01G 19/44

(52) U.S. Cl. CPC G01G 17/04; A61B 5/20; G01G 19/44

(57) ABSTRACT

A urination amount measuring apparatus includes: a body weight measuring unit (100); a urination amount calculating unit (276) configured to obtain a urination amount from measurement results of the body weight measuring unit; an assistance setting unit (260, 272) configured to set an assistance mode when a subject uses the body weight measuring unit; a storage unit (277) configured to store a urination amount measurement procedure corresponding to the assistance mode; and an output unit (210, 274) (220, 275) configured to read the urination amount measurement procedure corresponding to the assistance mode set by the assistance setting unit from the storage unit and output the urination amount measurement procedure. Accordingly, it is possible to obtain an accurate urination amount.

### TECHNICAL FIELD

The present invention relates to a urination amount measuring apparatus.

### BACKGROUND ART

There is a proposed urination amount management system for managing a daily urination amount in order to manage the physical condition of a patient having a disorder in the urine accumulation function or the urination function or to confirm the effect of treatment (Patent Literature 1). This system includes a body weight measuring unit and a urination amount management terminal connected to the body weight measuring unit through a communication line. When receiving body weight values before and after urination transmitted from the body weight measuring unit through the communication line, the urination amount management terminal calculates a difference between these body weight values as the urination amount. The patient only needs to get on the body weight measuring unit before and after urination to measure his/her body weight in order to enable measurement of the urination amount.

Many patients with disorders in the urine accumulation function or the urination function are elderly, and particularly in a case of an elderly patient with weak legs, it is difficult for the patient to get on and off the body weight measuring unit by him/herself. On the other hand, Patent Literature 2 proposes a body weight scale with a handrail (Patent Literature 2). In this body weight scale, the handrail is provided on a load plate whose four corners are equipped with load cells for detecting the load.

### CITATION LIST

### Patent Literature

Patent Literature 1: JP 2018-175349 A
Patent Literature 2: JP 2019-002734 A

### SUMMARY OF INVENTION

### Technical Problem

The body weight scale described in Patent Literature 2 can accurately measure the body weight of a patient even when the body weight is measured if the patient on the body weight scale holds the handrail. On the other hand, a patient may sometimes get on the body weight scale using a handrail provided separately from the body weight scale or with his/her body being supported by an assistant such as a nurse. In such a case, there is a problem that, if the body weight is measured while the patient holds the handrail or while the assistant supports his/her body, the body weight of the patient cannot be accurately measured, and as a result, an accurate urination amount cannot be calculated.

An object of the present invention is to enable obtaining an accurate urination amount in a urination amount measuring apparatus that calculates a urination amount from body weight measurement values before and after urination.

### Solution to Problem

A urination amount measuring apparatus according to the present invention made to solve the above problem includes:
a body weight measuring unit;
a urination amount calculating unit configured to obtain a urination amount from measurement results of the body weight measuring unit;
an assistance setting unit configured to set an assistance mode when a subject uses the body weight measuring unit;
a storage unit configured to store a urination amount measurement procedure corresponding to the assistance mode; and
an output unit configured to read the urination amount measurement procedure corresponding to the assistance mode set by the assistance setting unit from the storage unit and output the urination amount measurement procedure.

### Advantageous Effects of Invention

According to the urination amount measuring apparatus of the present invention, a urination amount measurement procedure corresponding to an assistance mode when body weight measurement is performed is output. Therefore, it is possible to notify a subject of an appropriate urination amount measurement procedure, making it possible to obtain an accurate urination amount.

### BRIEF DESCRIPTION OF DRAWINGS

FIG. 1 A schematic overall configuration diagram of a urination amount management system according to an embodiment of the present invention.

FIG. 2 A schematic block diagram of a body weight measuring unit and an output device constituting a urination amount measuring apparatus.

FIG. 3 A diagram showing an example of display image on an output device for explaining a flow of urination amount measurement processing in a case assistance is absent.

FIG. 4 A diagram showing an example of display image on the output device for explaining a flow of urination amount measurement processing in a case where assistance is present.

FIG. 5 A diagram showing an example of a display image on the output device when setting presence or absence of assistance and a delay time.

### DESCRIPTION OF EMBODIMENTS

Hereinafter, an embodiment embodying the present invention will be described with reference to the drawings.

### [Overall Configuration of Urination Amount Measuring Apparatus]

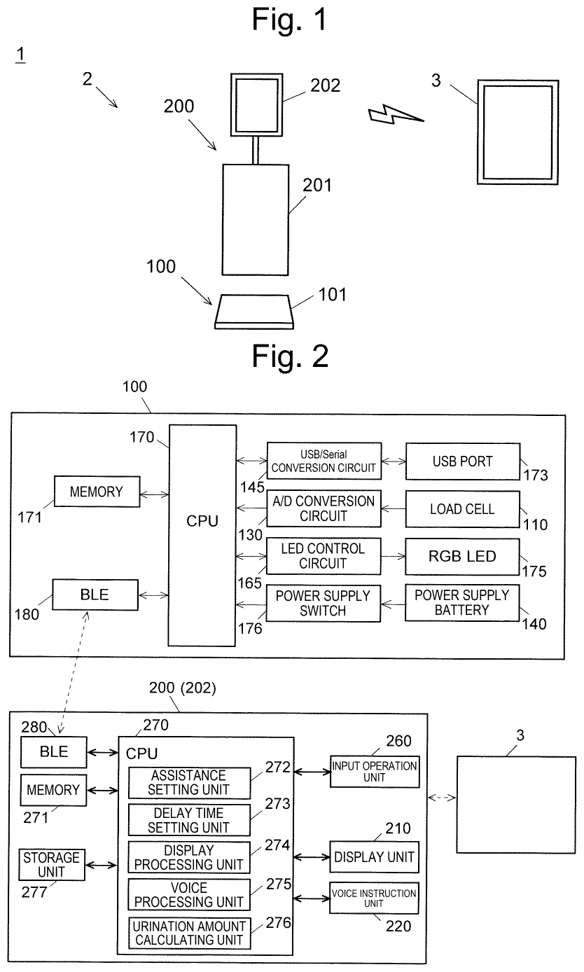

FIG. 1 is a diagram schematically showing a urination amount management system 1 according to the present embodiment. FIG. 2 is a schematic block diagram of the urination amount management system 1. This urination amount management system 1 includes a urination amount measuring apparatus 2 and a manager terminal 3. The urination amount management system 1 is installed in a medical institution such as a hospital, a home of a subject, or the like.

The urination amount measuring apparatus 2 includes a body weight measuring unit 100 and an output device 200. The body weight measuring unit 100 and the output device 200 include short-range wireless communication circuits (hereinafter, simply referred to as “communication circuit”) 180 and 280, respectively, and are configured to be able to wirelessly communicate with each other. The manager terminal 3 is, for example, a desktop-type, notebook-type, or tablet-type personal computer, and is connected to the output device 200 of the urination amount measuring apparatus 2 by short-range wireless communication. Note that the manager terminal 3 and the output device 200 may be connected via a local network in a facility where the urination amount management system 1 is installed or the Internet.

### [Configuration of Body Weight Measuring Unit]

The body weight measuring unit 100 includes a flat housing 101, and is mounted with a central processing unit (CPU) 170, a communication circuit 180, a power supply control circuit 160, an LED control circuit 165, an A/D conversion circuit 130, a USB/serial conversion circuit 145, a memory (read only memory (ROM) and random access memory (RAM)) 171, a power supply battery 140, an LED 175, and a load cell 110 housed in the housing 101.

The load cell 110 is a type of weight sensor in which distortion generated according to the body weight of the subject on the housing 101 appears as a change in resistance value. A change in the resistance value is output as a voltage signal or an electric current signal from the load cell 110. The signal output from the load cell 110 is converted into a digital value by the A/D conversion circuit 130 and then input to the CPU 170.

The USB/serial conversion circuit 145 converts various signals input and output from a USB port 173 into serial data and inputs the serial data to the CPU 170. The ROM houses a program in which a processing procedure of the CPU 170 is described, and the CPU 170 functions as a control unit by developing and executing the program in a RAM or the like. The ROM also houses various tables (maps) used for various arithmetic operations. The CPU 170 executes various types of processing in the body weight measuring unit 100 according to these programs and tables. The various types of processing are not limited to being executed by software, and can be executed by dedicated hardware (electronic circuit).

The body weight measuring unit 100 further includes a power supply switch 176 for turning on/off the body weight measuring unit 100. Even when the body weight measuring unit 100 is turned on by the power supply switch 176, the body weight measuring unit 100 is in a sleep state in principle when the body weight is not measured in order to realize low power consumption.

The CPU 170 has high processing capability to enable high-speed arithmetic operation up to a floating point in order to realize weight measurement. Therefore, if the load cell 110 and the processing circuit at the subsequent stage are always in a standby state in order to detect the load, power consumption is large. Accordingly, in the urination amount measuring apparatus 2 of the present embodiment, in the sleep state, some functions of the communication circuit 180 of the body weight measuring unit 100 and the CPU 170 that controls the communication circuit 180 are activated with low power. When the CPU 170 receives a wireless communication request from the output device 200, the communication circuit 180 is activated, and communication with the output device 200 is established. When the communication is established, the CPU 170 is fully activated. As the communication circuit 180, for example, it is preferable to use one conforming to a low power standard such as Bluetooth (registered trademark) Low Energy. The CPU 170 displays the operating state of the body weight measuring unit 100 to the outside by the LED 175 which is an indicator of RGB.

In the body weight measuring unit 100, after the communication with the output device 200 is established, the CPU 170 changes from the low-power activation state to a state in which the high-performance arithmetic operation can be performed, and the power supply control circuit 160 is energized with another unit such as the load cell 110. At the time of body weight measurement, the CPU 170 starts signal arithmetic operation of the load cell 110, and thus power consumption increases.

With the above configuration, it is possible to limit the power consumption of the body weight measuring unit 100 in the sleep state to the power consumption of the CPU 170 in the low power state. In the urination amount management system 1, since the body weight measurement is performed twice, before and after urination, each time the patient urinates, the power consumption of the body weight measuring unit 100 is significantly increased as compared with the case where the body weight measurement is performed once a day, for example. In the body weight measuring unit 100 of the present embodiment, since the power consumption in the sleep state is reduced, the life of the battery 140 can be dramatically extended.

### [Configuration of Output Device]

The output device 200 includes a base 201 and a device main body 202 attached onto the base. The device main body 202 includes, for example, a tablet terminal detachably attached to the base 201. The device main body 202 includes a CPU 270, a memory 271, a storage unit 277, an input operation unit 260 including a touch panel or a button for receiving an input operation, a display unit 210 including a display panel for displaying a screen, a voice instruction unit 220 including a speaker, and a communication circuit 280. The CPU 270 further includes, as functional blocks, an assistance setting unit 272, a delay time setting unit 273, a display processing unit 274, a voice processing unit 275, and a urination amount calculating unit 276. The output device 200 may be integrated with the device main body 202.

The assistance setting unit 272 sets an assistance mode (type of assistance, presence or absence of assistance, and the like) when the subject uses the body weight measuring unit 100. In the present embodiment, the assistance setting unit 272 sets whether or not a handrail is installed in the vicinity of the body weight measuring unit 100 (one or both of the left side and the right side of the body weight measuring unit 100), the handrail being usable when getting on and off the housing 101 of the body weight measuring unit 100. Note that this handrail is provided separately from the body weight measuring unit 100, and may be a dedicated handrail installed for the urination amount measuring apparatus 2, or a handrail installed in a hallway or the like of a hospital may be used. The delay time setting unit 273 sets a time from when the body weight measuring unit 100 detects the body weight of the subject to when the body weight measurement is started (time from input of signal output from the load cell 110 to the CPU 170 of the body weight measuring unit 100 to start of arithmetic operation of the signal) (hereinafter, referred to as “delay time”). The presence or absence of the handrail and the delay time are set in advance by a manager or a worker such as a nurse requested by the manager operating the input operation unit 260 (hereinafter, referred to as “manager or the like”). The input operation unit 260 and the assistance setting unit 272 in the present embodiment correspond to an “assistance setting unit” of the present invention, and the input operation unit 260 and the delay time setting unit 273 correspond to a “delay time setting unit” (described later) of the present invention.

The storage unit 277 stores different urination amount measurement procedures according to the presence or absence of a handrail. Specifically, a display image and a voice message representing the contents of each processing step of the urination amount measurement procedure are stored together with the order of each processing step.

The display processing unit 274 reads the urination amount measurement procedure corresponding to the presence or absence of the handrail set by the assistance setting unit 272 from the storage unit 277, and displays the urination amount measurement procedure on the display unit 210 as a display image. The voice processing unit 275 reads a urination amount measurement procedure corresponding to the presence or absence of the handrail set by the assistance setting unit 272 from the storage unit 277, and outputs the urination amount measurement procedure to the voice instruction unit 220 as a voice message. The display processing unit 274 and the display unit 210, and the voice processing unit 275 and the voice instruction unit 220, in the present embodiment each correspond to an “output unit” of the present invention.

The urination amount calculating unit 276 calculates the urination amount from the body weight values measured by the body weight measuring unit 100. Specifically, the urination amount is obtained by subtracting the body weight value measured after urination from the body weight value measured before urination. If necessary, the body weight value may be converted into a volume using a standard density of urine.

Application software dedicated to the urination amount management system 1 is housed in the memory 271, and the CPU 270 executes the software to make the assistance setting unit 272, the delay time setting unit 273, the display processing unit 274, the voice processing unit 275, and the urination amount calculating unit 276 function.

### [Urination Amount Measurement Processing]

Next, urination amount measurement processing in the urination amount measuring apparatus 2 will be described.

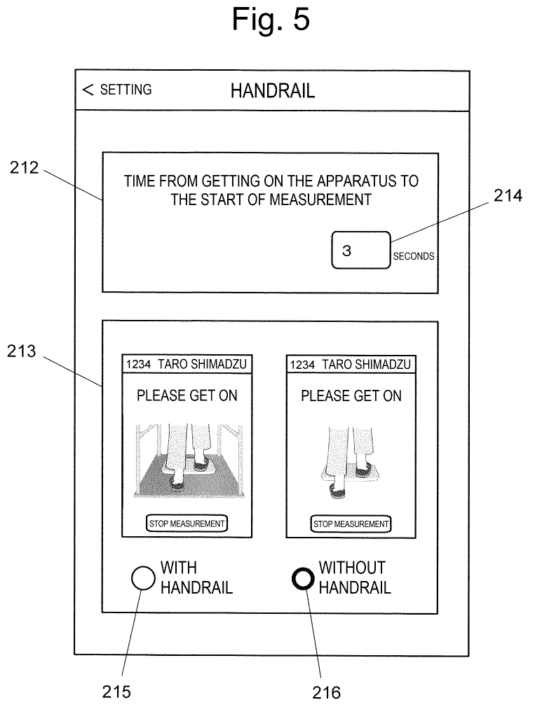

First, before starting to use the urination amount measuring apparatus 2, the manager or the like of the urination amount management system 1 sets conditions necessary for the urination amount measurement processing. FIG. 5 shows an example of an image (manager screen) shown on the display unit 210 of the output device 200 for the manager or the like to set conditions.

For example, FIG. 5 is a setting screen for setting whether or not there is a “handrail” in the vicinity of the body weight measuring unit 100. This setting screen includes two rectangular areas 212 and 213 disposed vertically, and the above-described delay time is set in the area 212, and the presence or absence of the handrail is set in the area 213. Therefore, the area 212 displays an explanatory sentence regarding the delay time (“Time from getting on the apparatus to the start of measurement”) and a graphical user interface (GUI) input field 214 for inputting the delay time. In addition, the area 213 vertically displays an example of display image corresponding to the presence and absence of a handrail (“with handrail” and “without handrail”) and texts indicating the presence and absence of a handrail. GUI radio buttons 215 and 216 for selecting “with handrail” and “without handrail” are displayed on the left side of the texts, respectively.

In a state where the setting screen shown in FIG. 5 is displayed on the display unit 210, the manager or the like touches the GUI radio button 215 or 216 in the area 213 on the touch panel 260, whereby the presence or absence of the handrail is selected. FIG. 5 shows a state in which “without handrail” is selected. The assistance setting unit 272 sets the presence or absence of the handrail based on this selection. In addition, the manager or the like touches the GUI input field 214 in the area 212 on the touch panel 260, and inputs the delay time into the input field 214 using a displayed software keyboard or the like. The delay time setting unit 273 sets the input value as a delay time and transmits the delay time to the CPU 170 of the body weight measuring unit 100.

The delay time is set in a range of, for example, 0 seconds to 30 seconds. For a young subject who is not difficult to move up and down the housing 101 of the body weight measuring unit 100, a subject having a urinary urgency symptom, and the like, it is preferable that the delay time is set to be short so that measurement is started immediately. On the other hand, in a case where the subject is elderly and it takes time from when the subject starts to get on the housing 101 to when the subject stands still, it is preferable that the delay time is set to be rather long. If the delay time is set to be rather long, it is possible to obtain an accurate body weight measurement value even in a case where it takes time from when the subject starts to get on the housing 101 to when the subject finishes getting on, or in a case where it takes time from when the subject gets on the housing 101 to when the subject stands still.

In addition, the body weight measuring unit 100 has a function of determining whether or not the output signal from the load cell 110 stabilizes by the CPU 170 determining whether or not the fluctuation amplitude of the signal output from the load cell 110 is within a predetermined range. In a case where the signal does not stabilize until a preset time elapses after the CPU 170 receives the signal from the load cell 110, the body weight measuring unit 100 may determine that there is a measurement error and stop the measurement. In the body weight measuring unit 100 as described above, by making the delay time longer than the set time, it is possible to start the arithmetic operation of the body weight of the subject after the output signal of the load cell 100 stabilizes.

Next, an action when the body weight of the subject is measured using the body weight measuring unit 100 and the output device 200 will be described. Note that, it is assumed that the power supply switch 176 has powered on the body weight measuring unit 100.

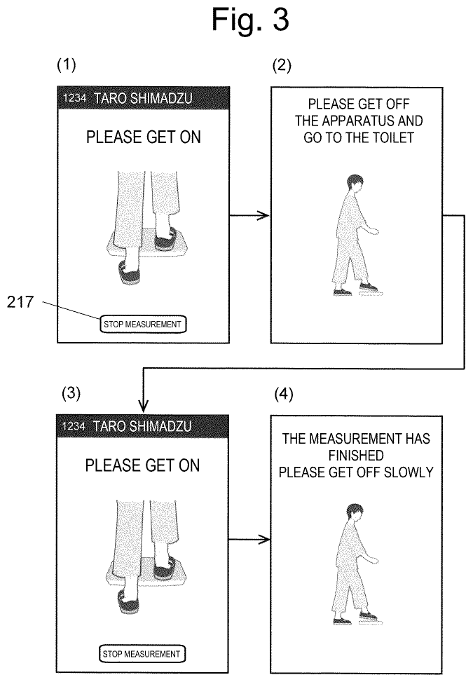

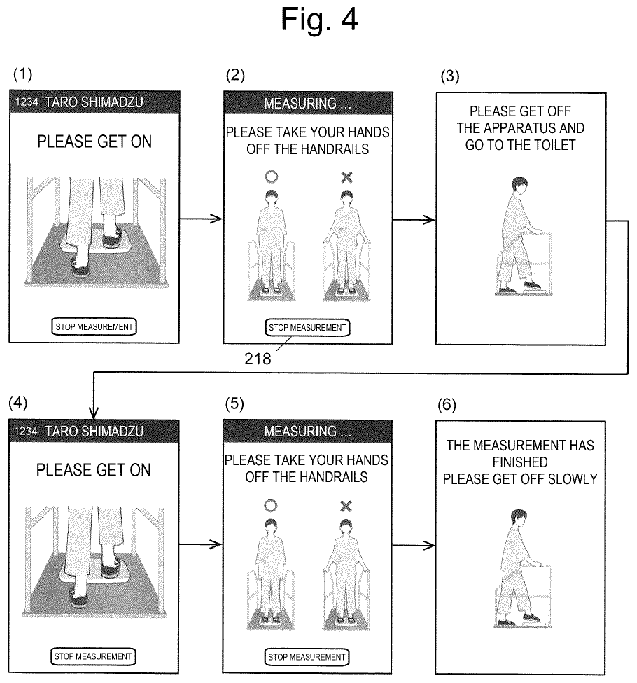

FIG. 3 is an example of a display image shown on the display unit 210 of the output device 200 in a case where “without handrail” is set, and FIG. 4 is an example of a display image shown on the display unit 210 of the output device 200 in a case where “with handrail” is set.

The subject who uses the body weight measuring unit 100 and the output device 200 first touches the touch panel 260 of the output device 200. Then, a name selection screen (not shown) is displayed on the display unit 210. The subject touches and selects his/her name displayed on the display unit 210 on the touch panel 260, and presses a measurement start button. As a result, discrimination information of the subject is input from the input operation unit 260 to the CPU 270.

When the CPU 270 receives the discrimination information, the CPU 270 activates the communication circuit 280 and transmits a wireless communication request to the body weight measuring unit 100. The body weight measuring unit 100 that has received the wireless communication request activates the communication circuit 180 to establish communication with the output device 200. When the communication is established, the CPU 270 of the output device 200 transmits a signal of “measurement preparation instruction” to the body weight measuring unit 100. When the body weight measuring unit 100 receives the signal of “measurement preparation instruction”, the CPU 170 is fully activated, and a circuit necessary for measurement such as the load cell 110 transitions from the sleep state to a measurable state. After that, the CPU 170 returns a signal of “measurement preparation completed” to the output device 200. When the CPU 270 of the output device 200 receives the signal of “measurement preparation completed”, the CPU 270 executes action instruction screen display processing and voice instruction processing on the display unit 210 and the voice instruction unit 220. First, processing in a case where “without handrail” is set will be described with reference to FIG. 3.

When the CPU 270 receives the signal of “measurement preparation completed”, the display processing unit 274 reads a screen with contents instructing an action to be performed next by the subject (action instruction screen) from the storage unit 277, and displays the screen on the display unit 210 (FIG. 3 (1)).

The action instruction screen of FIG. 3 (1) displays a text message (“Please get on”) instructing the subject to get on the housing 101 of the body weight measuring unit 100 in order to measure the body weight, an image showing a state in which a person is about to get on the housing 101, and a GUI button 217 for confirming an intention to stop the measurement. The image may be a still image or a moving image. The GUI button 217 is formed by a rectangular frame and texts “Stop measurement” displayed in the frame. In addition, discrimination information (discrimination number, name of the subject) received by the CPU 270 from the input operation unit 260 is displayed in an upper portion of the action instruction screen.

In addition, when the CPU 270 of the output device 200 receives the signal of “measurement preparation completed” from the body weight measuring unit 100, the voice processing unit 275 reads, from the storage unit 277, a voice message with contents instructing an action to be performed next, and outputs the voice message from the voice instruction unit 220.

Therefore, the subject can decide the action to be performed next by looking at the text message or the image displayed on the screen of the display unit 210 or listening to the voice message output from the voice instruction unit 220. In this manner, since the action to be performed next is instructed by both the visual information of the text message and the image and the auditory information of the voice message, use is easy even in a case where either or both of the eyesight and the hearing of the subject are deteriorated or there is a problem in the cognitive function. In addition, since the content of the action instruction is expressed by the image, use is easy even for infants and foreign patients who cannot understand the instruction content of texts and voices.

Note that, in a case where the signal of “measurement preparation completed” is not transmitted from the body weight measuring unit 100 even when the predetermined time elapses after the output device 200 transmits the signal of “measurement preparation instruction”, the CPU 270 transmits the signal of “measurement preparation instruction” again. When the signal of “measurement preparation completed” is not transmitted from the body weight measuring unit 100 even after the processing of transmitting the signal of “measurement preparation instruction” is repeated a plurality of times (for example, 10 times), an error screen is displayed on the display unit 210.

On the other hand, the CPU 170 of the body weight measuring unit 100 that has transitioned from the sleep state to the measurable state observes the change in the resistance value of the load cell 110 for a certain period of time based on a digital value obtained from the A/D conversion circuit 130.

When the subject gets on the housing 101 of the body weight measuring unit 100, the digital value representing a change in the resistance value of the load cell 110 is input from the A/D conversion circuit 130 to the CPU 170. The CPU 170 detects from the digital value that the subject is in a state of being on the housing 101.

The CPU 170 detects that the subject is in a state of being on the housing 101 and transmits a signal of “load variation detected” to the output device 200. After that, when the time set by the delay time setting unit 273 has elapsed, the CPU 270 of the output device 200 transmits a signal of “measurement start instruction” to the CPU 170 of the body weight measuring unit 100. Upon receiving the signal of “measurement start instruction”, the CPU 170 starts arithmetic operation of the signal of the load cell 110. The CPU 170 calculates the body weight measurement value of the subject while performing averaging processing, stabilization determination, and the like of the digital value input from the load cell 110. As a result, even in a case where it takes time from when the subject starts to get on the housing 101 to when the subject stands still, it is possible to accurately perform the body weight measurement. The obtained body weight measurement value is stored in the memory 171 of the body weight measuring unit 100.

When the calculation of the body weight measurement value of the subject is finished, the CPU 170 transmits a signal of “weight measurement completed” to the output device 200. When the CPU 270 of the output device 200 receives the signal of “weight measurement completed”, the display processing unit 274 reads a next action instruction screen from the storage unit 277 and displays the next action instruction screen on the display unit 210 (FIG. 3 (2)). This screen displays a text message (“Please get off the apparatus and go to the toilet”) for explaining the action to be performed after the measurement is finished and an image representing a state in which a person is about to get down from the housing 101 of the body weight measuring unit 100. In addition, when the CPU 270 receives a signal of “weight measurement completed”, the voice processing unit 275 reads a voice message with contents explaining a next action from the storage unit 277, and outputs the voice message to the voice instruction unit 220.

When the subject gets off the housing 101 of the body weight measuring unit 100, the digital value input from the A/D conversion circuit 130 rapidly decreases. The CPU 170 that has detected from the change in the digital value that the subject has gotten off the housing 101 transmits a signal of “load variation detected” to the output device 200. In addition, the CPU 170 reads the body weight measurement value when the subject is on the housing 101 from the memory 171, and subtracts, from the body weight measurement value, the body weight measurement value calculated from the digital value when the subject is not on the housing 101 to obtain the body weight value of the subject. The obtained body weight value is retained in the memory 171 and transmitted to the output device 200 as body weight value data before urination.

The CPU 270 of the output device 200 that has received the body weight value data before urination from the body weight measuring unit 100 retains the body weight value data in the memory 271, and transmits a signal of “body weight value data reception completed” to the body weight measuring unit 100. The CPU 170 of the body weight measuring unit 100 repeats the transmission processing of the body weight value data a plurality of times (for example, 10 times) until receiving the signal of “body weight value data reception completed” from the CPU 270 of the output device 200.

The CPU 170 of the body weight measuring unit 100 that has received the signal of “body weight value data reception completed” from the output device 200 finishes the transmission processing of the body weight value data. After that, the output device 200 transmits a signal of “measurement finished” to the body weight measuring unit 100, and the body weight measuring unit 100 that has received the signal of “measurement finished” transmits a signal of “measurement finish confirmed” to the output device 200. The output device 200 that has received the signal of “measurement finish confirmed” disconnects communication with the body weight measuring unit 100. When the communication is disconnected, the body weight measuring unit 100 and the output device 200 enter the sleep state.

When a predetermined time elapses after the output device 200 enters the sleep state, the CPU 270 activates the communication circuit 280 to transmit a wireless communication request to the body weight measuring unit 100 and establishes communication with the body weight measuring unit 100. When the communication is established, the CPU 270 of the output device 200 transmits a signal of “measurement preparation instruction” to the body weight measuring unit 100. When the body weight measuring unit 100 receives the signal of “measurement preparation instruction”, the body weight measuring unit 100 transitions from the sleep state to the measurable state. After that, the CPU 170 returns a signal of “measurement preparation completed” to the output device 200.

When the CPU 270 of the output device 200 receives the signal of “measurement preparation completed”, the display processing unit 274 reads a next action instruction screen from the storage unit 277 and displays the screen on the display unit 210 (FIG. 3 (3)). This screen is the same as that in FIG. 3 (1), and a text message (“Please get on”) instructing the subject to get on the housing 101 of the body weight measuring unit 100 is displayed.

In addition, the voice processing unit 275 reads, from storage unit 277, a voice message with contents instructing an action to be performed next, and outputs the voice message to the voice instruction unit 220.

Similarly to the body weight measurement before urination, the CPU 170 of the body weight measuring unit 100 calculates the body weight measurement value of the subject after urination and stores the body weight measurement value in the memory 171. When the calculation of the body weight measurement value of the subject is finished, the CPU 170 transmits a signal of “weight measurement completed” to the output device 200. When the CPU 270 of the output device 200 receives the signal of “weight measurement completed”, the display processing unit 274 reads, from the storage unit 277, a screen representing that the measurement is finished and displays the screen on the display unit 210 (FIG. 3 (4)). This screen displays a text message explaining an action to be performed after the measurement is finished (“The measurement has finished. Please get off slowly”) and an image representing a state in which a person is about to get down from the housing 101. In addition, the voice processing unit 275 reads, from the storage unit 277, a voice message with contents instructing an action to be performed after the measurement is finished, and outputs the voice message to the voice instruction unit 220.

The CPU 170 that has detected from the change in the digital value that the subject has gotten off the housing 101 transmits a signal of “load variation detected” to the output device 200. In addition, the CPU 170 reads the body weight measurement value when the subject is on the housing 101 from the memory 171, and subtracts, from the body weight measurement value, the body weight measurement value calculated from the digital value when the subject is not on the housing 101 to obtain the body weight value of the subject. The obtained body weight value is transmitted to the output device 200 as body weight value data after urination.

The CPU 270 of the output device 200 that has received the body weight value data after urination from the body weight measuring unit 100 retains the body weight value data in the memory 271, and transmits a signal of “body weight value data reception completed” to the body weight measuring unit 100. The CPU 170 of the body weight measuring unit 100 that has received the signal of “body weight value data reception completed” from the output device 200 finishes the transmission processing of the body weight value data. The output device 200 transmits a signal of “measurement finished” to the body weight measuring unit 100, and the body weight measuring unit 100 that has received the signal of “measurement finished” transmits a signal of “measurement finish confirmed” to the output device 200. When the output device 200 receives the signal of “measurement finish confirmed”, the urination amount calculating unit 276 reads the body weight value before urination retained in the memory 271, and calculates the urination amount by subtracting the body weight value after urination from the body weight value before urination. The calculated urination amount is stored in the memory 271. After that, the CPU 270 transmits the urination amount stored in the memory 271 to the manager terminal 3 in association with the discrimination information of the subject, and finishes the urination amount measurement processing.

Next, urination amount measurement processing in a case where “with handrail” is set will be described with reference to FIG. 4. In FIG. 4, handrails are shown in the images of various displayed screens. Note that the following description will be appropriately simplified for a portion overlapping with the content in a case where “without handrail” is set.

As described above, the subject who uses the body weight measuring unit 100 and the output device 200 first touches the touch panel 260 of the output device 200, selects his/her name on the touch panel 260 on the name selection screen displayed, and presses the measurement start button. As a result, discrimination information of the subject is input from the input operation unit 260 to the CPU 270.

When the CPU 270 receives the discrimination information, the CPU 270 activates the communication circuit 280 to transmit a wireless communication request to the body weight measuring unit 100 and establishes communication with the body weight measuring unit 100. When the communication is established, the CPU 270 of the output device 200 transmits a signal of “measurement preparation instruction” to the body weight measuring unit 100. When the body weight measuring unit 100 receives the signal of “measurement preparation instruction”, the body weight measuring unit 100 transitions from the sleep state to the measurable state. After that, the CPU 170 returns a signal of “measurement preparation completed” to the output device 200.

When the CPU 270 receives the signal of “measurement preparation completed”, the display processing unit 274 reads, from the storage unit 277, a screen instructing an action to be performed next by the subject (action instruction screen), and displays the screen on the display unit 210 (FIG. 4 (1)). FIG. 4 (1) is the same as FIG. 3 (1) except that handrails are shown in the image, and displays a text message (“Please get on”) instructing the subject to get on the housing 101 of the body weight measuring unit 100 for measuring the body weight. In addition, the voice processing unit 275 outputs a voice message with contents instructing an action to be performed next from the voice instruction unit 220.

On the other hand, the CPU 170 of the body weight measuring unit 100 that has transitioned from the sleep state to the measurable state observes the change in the resistance value of the load cell 110 for a certain period of time. Upon detecting that the subject is in a state of being on the housing 101, the CPU 170 transmits a signal of “load variation detected” to the output device 200. When the time set by the delay time setting unit 273 has elapsed from when the output device 200 has received the signal of “load variation detected”, the CPU 270 of the output device 200 transmits a signal of “measurement start instruction” to the CPU 170 of the body weight measuring unit 100. As a result, arithmetic operation of the signal of the load cell 110 by the CPU 170 is started.

In addition, when the time set by the delay time setting unit 273 has elapsed from when the output device 200 has received the signal of “load variation detected”, the display processing unit 274 of the output device displays the next action instruction screen on the display unit 210 (FIG. 4 (2)). This screen displays texts indicating that the body weight is being measured (“Measuring . . . ”), a text message indicating contents to be noted during the measurement (“Please take your hands off the handrails”), an image indicating a person standing upright on the housing 101 with the hands off the handrails and a person standing on the housing 101 with holding the handrails, and a GUI button 218 for confirming an intention to stop the measurement. In the image of FIG. 4 (2), a symbol of ∘ is displayed on a person with the hands off the handrails, and a symbol of x is displayed on a person with holding the handrails.

The CPU 170 of the body weight measuring unit 100 calculates the body weight measurement value of the subject while performing stabilization determination and averaging processing of the digital value input from the load cell 110, and stores the weight measurement value in the memory 171. When the calculation of the body weight measurement value of the subject is finished, the CPU 170 transmits a signal of “weight measurement completed” to the output device 200. When the CPU 270 of the output device 200 receives the signal of “weight measurement completed”, the display processing unit 274 switches the screen of the display unit 210 to the next action instruction screen (FIG. 4 (3)). FIG. 4 (3) is the same as FIG. 3 (2) except that a handrail is shown in the image, and displays a text message (“Please get off the apparatus and go to the toilet”) for explaining the action to be performed after the body weight measurement is finished. At the same time, a voice message with the same contents as this text message is output from the voice instruction unit 220.

The CPU 170 that has detected from the change in the digital value that the subject has gotten off the housing 101 of the body weight measuring unit 100 transmits a signal of “load variation detected” to the output device 200. In addition, the CPU 170 reads the body weight measurement value when the subject is on the housing 101 from the memory 171, and subtracts, from the body weight measurement value, the body weight measurement value calculated from the digital value when the subject is not on the housing 101 to obtain the body weight value of the subject. The obtained body weight value is retained in the memory 171 and transmitted to the output device 200 as body weight value data before urination.

The CPU 270 of the output device 200 that has received the body weight value data before urination from the body weight measuring unit 100 retains the body weight value data in the memory 271, and transmits a signal of “body weight value data reception completed” to the body weight measuring unit 100. The CPU 170 of the body weight measuring unit 100 that has received the signal of “body weight value data reception completed” from the output device 200 finishes the transmission processing of the body weight value data. After that, the output device 200 transmits a signal of “measurement finished” to the body weight measuring unit 100, and the body weight measuring unit 100 that has received the signal of “measurement finished” transmits a signal of “measurement finish confirmed” to the output device 200. The output device 200 that has received the signal of “measurement finish confirmed” disconnects communication with the body weight measuring unit 100. When the communication is disconnected, the body weight measuring unit 100 and the output device 200 enter the sleep state.

When a predetermined time elapses after the output device 200 enters the sleep state, the CPU 270 activates the communication circuit 280 to transmit a wireless communication request to the body weight measuring unit 100 and establishes communication with the body weight measuring unit 100. When the communication is established, the CPU 270 of the output device 200 transmits a signal of “measurement preparation instruction” to the body weight measuring unit 100. When the body weight measuring unit 100 receives the signal of “measurement preparation instruction”, the body weight measuring unit 100 transitions from the sleep state to the measurable state. After that, the CPU 170 returns a signal of “measurement preparation completed” to the output device 200.

When the CPU 270 of the output device 200 receives the signal of “measurement preparation completed”, the display processing unit 274 displays the next action instruction screen on the display unit 210 (FIG. 4 (4)). FIG. 4 (4) is the same as FIG. 4 (1), and displays a text message instructing the subject to get on the housing 101 of the body weight measuring unit 100 for measuring the body weight. In addition, a voice message with the same contents as this text message is output from the voice instruction unit 220.

On the other hand, the CPU 170 of the body weight measuring unit 100 that has transitioned from the sleep state to the measurable state observes the change in the resistance value of the load cell 110 for a certain period of time. Upon detecting that the subject is in a state of being on the housing 101, the CPU 170 transmits a signal of “load variation detected” to the output device 200. When the time set by the delay time setting unit 273 has elapsed from when the output device 200 has received the signal of “load variation detected”, the CPU 270 of the output device 200 transmits a signal of “measurement start instruction” to the CPU 170 of the body weight measuring unit 100. As a result, arithmetic operation of the signal of the load cell 110 by the CPU 170 is started.

In addition, when the time set by the delay time setting unit 273 has elapsed from when the output device 200 has received the signal of “load variation detected”, the display processing unit 274 of the output device displays the next action instruction screen on the display unit 210 (FIG. 4 (5)). FIG. 4 (5) is the same as FIG. 4 (2), and displays a text message for explaining cautions during measurement. In addition, a voice message with the same contents as this text message is output from the voice instruction unit 220.

Similarly to the body weight measurement before urination, the CPU 170 of the body weight measuring unit 100 calculates the body weight measurement value of the subject. When the calculation of the body weight measurement value of the subject is finished, the CPU 170 transmits a signal of “weight measurement completed” to the output device 200. When the CPU 270 of the output device 200 receives the signal of “weight measurement completed”, the display processing unit 274 switches the screen of the display unit 210 to a screen indicating that the measurement is finished (FIG. 4 (6)). FIG. 4 (6) is the same as FIG. 3 (4) except that a handrail is shown in the image, and displays a text message (“The measurement has finished. Please get off slowly”) explaining the action to be performed after the measurement is finished. In addition, a voice message with the same contents as this text message is output from the voice instruction unit 220.

The CPU 170 that has detected from the change in the digital value that the subject has gotten off the housing 101 transmits a signal of “load variation detected” to the output device 200. In addition, the body weight value of the subject after urination is obtained in the same manner as before urination. The obtained body weight value is retained in the memory 171 and transmitted to the output device 200 as body weight value data after urination.

The CPU 270 of the output device 200 that has received the body weight value data after urination from the body weight measuring unit 100 retains the body weight value data in the memory 271, and transmits a signal of “body weight value data reception completed” to the body weight measuring unit 100. The CPU 170 of the body weight measuring unit 100 that has received the signal of “body weight value data reception completed” from the output device 200 finishes the transmission processing of the body weight value data. After that, the output device 200 transmits a signal of “measurement finished” to the body weight measuring unit 100, and the body weight measuring unit 100 that has received the signal of “measurement finished” transmits a signal of “measurement finish confirmed” to the output device 200. When the output device 200 receives the signal of “measurement finish confirmed”, the urination amount calculating unit 276 reads the body weight value before urination retained in the memory 271, and calculates the urination amount by subtracting the body weight value after urination from the body weight value before urination. The calculated urination amount is stored in the memory 271. After that, the CPU 270 transmits the urination amount stored in the memory 271 to the manager terminal 3 in association with the discrimination information of the subject, and finishes the urination amount measurement processing.

### (Modifications)

The present invention is not limited to the above embodiment, and can be appropriately modified.

For example, in the above embodiment, as the setting of the assistance mode, whether or not the handrail is installed in the vicinity of the housing 101 of the body weight measuring unit 100 is set. However, for example, the assistance setting unit 272 may set whether or not there is an assistant who assists the subject to get on and off the housing 101. In such a case, the assistant is shown on the image displayed on the display unit 210 of the output device 200 instead of the handrail.

In addition, the present invention can also be applied to a case where measurement is performed in a state where the subject sits on the housing 101 instead of standing on the housing 101. For example, in a case where it is difficult for a subject who is a wheelchair user to take a standing position, it is conceivable that the measurement is performed when the subject gets on the housing 101 in a state of seating on the wheelchair. In addition, in a case of a subject who is not a wheelchair user but has difficulty in continuously taking a standing position, it is conceivable that a chair is placed on the housing 101 and measurement is performed in a state of seating on the chair. In such a case, it is conceivable to display, according to the use of the wheelchair or the chair (assistance mode), the posture at the time of getting on and off the housing 101, measurement, and the like on the display unit 210 of the output device 200. In these examples, as the body weight measuring unit 100, it is possible to use a unit including a housing 101 having a size on which a wheelchair or a chair can be placed and having a slope so that the wheelchair can easily get on and off, or a unit in which the housing 101 itself is a chair type.

In the above embodiment, the subject touches the touch panel 260, selects his/her name on the displayed name selection screen, and then presses the measurement start button, so that communication between the body weight measuring unit 100 and the output device 200 is established and the processing is started. However, the method for starting the processing is not limited to the above. For example, in a case where the urination amount measuring apparatus 2 further includes a barcode reader as a device for acquiring discrimination information of the subject and a barcoded wristband having discrimination information recorded for discriminating an individual is assigned to each subject, the processing may be started by the subject holding his/her wristband over the barcode reader.

Whether or not assistance by an assistant is necessary at the time of getting on and off the body weight measuring unit 100 varies depending on the health condition of the subject and the state of the legs and loins. Therefore, for example, the information on the presence or absence of the assistant may be recorded together with the discrimination information of the subject in the barcode of the wristband described above, and when the barcode reader reads the information on the presence or absence of the assistant, the information may be input to the assistance setting unit 272 to set the presence or absence of the assistant. In addition, the delay time may be similarly recorded in the barcode of the subject, and when the barcode reader reads the delay time, the delay time may be input to the delay time setting unit 273 to set the delay time. In addition, such information may be stored in advance in the memory 271 of the output device 200 for each subject, and the assistance setting unit 272 may read the information on the basis of the barcode reader or the discrimination information of the subject input from the input operation unit 260 to the CPU 270.

In the above embodiment, the body weight measuring unit 100 and the output device 200 are configured separately, but may be integrally formed. In addition, the output device 200 includes both the display unit 210 and the voice instruction unit 220, but may be configured to include any one of the display unit 210 and the voice instruction unit 220.

In the above embodiment, it has been described that the urination amount measuring apparatus 2 includes one body weight measuring unit 100 and one output device 200, but a plurality of output devices 200 may be provided. For example, a personal smartphone or the like of a subject may be used as the output device 200. In such a case, discrimination information for discriminating each terminal from another terminal is stored in advance in the memory 271 of the output device 200.

In the above embodiment, the output device 200 includes the urination amount calculating unit 276, but the urination amount calculating unit 276 may be included in the body weight measuring unit 100. In such a case, a urination amount measurement result is transmitted and received between the body weight measuring unit 100 of the urination amount measuring apparatus 2 and the manager terminal 3.

### [Modes]

A person skilled in the art can understand that the previously described illustrative embodiments are specific examples of the following modes of the present invention.

(Clause 1) A urination amount measuring apparatus according to one mode of the present invention includes:
a body weight measuring unit;
a urination amount calculating unit configured to obtain a urination amount from measurement results of the body weight measuring unit;
an assistance setting unit configured to set an assistance mode when a subject uses the body weight measuring unit;
a storage unit configured to store a urination amount measurement procedure corresponding to the assistance mode; and
an output unit configured to read the urination amount measurement procedure corresponding to the assistance mode set by the assistance setting unit from the storage unit and output the urination amount measurement procedure.

(Clause 2) In a urination amount measuring apparatus according to Clause 1,
the assistance mode may be presence or absence of assistance.

With a urination amount measuring apparatus according to Clause 1 or Clause 2, a urination amount measurement procedure corresponding to the assistance mode when body weight measurement is performed is output. Therefore, it is possible to appropriately instruct the subject on an action to be performed next, and to perform accurate body weight measurement. As a result, it is possible to accurately obtain the urination amount.

(Clause 3) In the urination amount measuring apparatus according to Clause 1 or Clause 2,
a delay time setting unit configured to set a delay time from when the body weight measuring unit detects body weight of the subject to when the body weight measuring unit starts body weight measurement of the subject may be further provided.

With a urination amount measuring apparatus according to Clause 3, the delay time can be set according to the time required from when the subject starts to get on the body weight measuring unit to when the subject stands still on the weight measuring unit. Therefore, the body weight measurement is started in a state where the subject stands still, and accurate body weight measurement can be performed.

(Clause 4) In the urination amount measuring apparatus according to Clause 3,
in a case where the assistance setting unit sets that the assistance is present,
the output unit may be configured to output a urination amount measurement procedure with contents notifying the subject to take his/her body away from the assistance when the delay time set by the delay time setting unit has elapsed.

With a urination amount measuring apparatus according to Clause 4, it is possible to prevent the body weight measurement from being started in a state where a part of the body weight of the subject is put on an assisting tool such as a handrail or an assistant.

(Clause 5) In the urination amount measuring apparatus according to Clause 1 to Clause 4,
the output unit may be configured to output the urination amount measurement procedure as an image on a display screen and/or as a voice message.

With a urination amount measuring apparatus according to Clause 5, it is possible to appropriately instruct the subject on an action to be performed next by visual information of the displayed screen and/or auditory information of the voice message.

### REFERENCE SIGNS LIST

1 . . . Urination Amount Management System
2 . . . Urination Amount Measuring Apparatus
100 . . . Body Weight Measuring Unit
101 . . . Housing
110 . . . Load Cell
130 . . . A/D Conversion Circuit
140 . . . Power Supply Battery
145 . . . USB/Serial Conversion Circuit
165 . . . LED Control Circuit
170 . . . CPU
171 . . . Memory
173 . . . USB Port
175 . . . LED
176 . . . Power Supply Switch
180 . . . Communication Circuit
200 . . . Output Device
201 . . . Base
202 . . . Device Main Body
210 . . . Display Unit
220 . . . Voice Instruction Unit
260 . . . Input Operation Unit
270 . . . CPU
271 . . . Memory
272 . . . Assistance Setting Unit
273 . . . Delay Time Setting Unit
274 . . . Display Processing Unit
275 . . . Voice Processing Unit
276 . . . Urination Amount Calculating Unit
277 . . . Storage Unit
280 . . . Communication Circuit
3 . . . Manager Terminal

1. A urination amount measuring apparatus comprising:
a body weight measuring unit;
a urination amount calculating unit configured to obtain a urination amount from measurement results of the body weight measuring unit;
an assistance setting unit configured to allow setting an assistance mode when a subject uses the body weight measuring unit;
a storage unit configured to store a urination amount measurement procedure corresponding to the assistance mode; and
an output unit configured to read the urination amount measurement procedure corresponding to the assistance mode set by the assistance setting unit from the storage unit and output the urination amount measurement procedure to the subject.

2. The urination amount measuring apparatus according to claim 1, wherein the assistance mode is presence or absence of assistance.

3. The urination amount measuring apparatus according to claim 2, wherein:
the body weight measuring unit comprises a load cell and a CPU; and
the body weight measuring unit further comprises a delay time setting unit configured to allow setting a delay time from when a signal output from the load cell is input to the CPU to when the CPU start to calculate the body weight measurement value of the subject based on the signal.

4. The urination amount measuring apparatus according to claim 3, wherein
in a case where the assistance setting unit sets that the assistance is present,
the output unit is configured to output a urination amount measurement procedure with contents notifying the subject to take his/her body away from the assistance when the delay time set by the delay time setting unit has elapsed.

5. The urination amount measuring apparatus according to claim 1, wherein the output unit is configured to output the urination amount measurement procedure as an image on a display screen and/or as a voice message.
